(19) United States
(12) Patent Application Publication
(10) Pub. No.: US 2017/0340283 A1
(43) Pub. Date: Nov. 30, 2017

(54) NEEDLE PROBE ARRAY AND METHODS REGARDING SAME

(71) Applicant: Ironwood Electronics, Inc., Eagan, MN (US)

(72) Inventors: Ilavarasan M. Palaniappa, Inver Grove Heights, MN (US); Mickiel P. Fedde, Eagan, MN (US); Brian M. Fedde, Burnsville, MN (US)

(21) Appl. No.: 15/163,023

(22) Filed: May 24, 2016

(51) Int. Cl. A61B 5/00; A61N 1/05

(52) U.S. Cl. CPC A61B 5/6848; A61N 1/0502

(57) ABSTRACT

A needle probe array may be configured to penetrate into biological tissue and may be coupled to an interface board. The needle probe array may include a proximal electrical interface region, a distal biological interface region, and a plurality of needle electrodes extending from the proximal electrical interface region to the distal biological interface region. Each needle electrode may include a head portion and a tip portion, the head portion may include contact surfaces and non-contact surfaces therebetween. The contact surfaces may contact and electrically couple the needle electrode to the interface board and the non-contact surfaces may lack contact with the interface board.

### BACKGROUND

The present disclosure relates generally to needle probe arrays that interact with and penetrate into biological tissue (e.g., skin) and to methods of providing (e.g., manufacturing) such needle probe arrays.

Skin is one example of a biological tissue. Skin is primarily made of two layers. The outer layer (epidermis) has a depth of approximately 100 micrometers. The inner layer (dermis) has a depth of approximately 3000 micrometers from the outer surface of the skin and is primarily composed of a network of fibrous protein known as collagen. The dermis contains vascular, nervous components, sweat glands, and hair follicles and is also electrically conducting.

Biological tissue, such as skin, may include defects (e.g., defects induced by aging, sun exposure, dermatological diseases, dramatic effects, etc.) that require diagnosis and/or treatment. Electrodes have been described that may be inserted or penetrate into the skin to be used as a stimulus electrode or a sensing electrode. For example, as described in U.S. Pat. No. 7,824,394 issued Nov. 2, 2010 and entitled “Method and Apparatus for Dermatological Treatment and Tissue Reshaping” electrodes may be used as stimulus electrodes to apply an electrical current to the skin to provide a variety of benefits, e.g., skin tightening and tissue remodeling. Further, as described therein, electrodes may be used to detect the presence of the nerve.

### SUMMARY

Although electrodes have previously been described as being usable to perform both as stimulus electrodes and sensing electrodes, when an array of such electrodes are used, various issues have become apparent. For example, it has been discovered that, even under the same conditions, variability of signals from or delivered by the various electrodes of the array exists. The present disclosure provides one or more solutions to reduce such variability.

An exemplary needle probe array to be coupled to an interface board defining a plurality of interface board openings described herein may include an electrical interface spacer located at a proximal electrical interface region of the needle probe array, a biological interface spacer located at a distal biological interface region of the needle probe array, and a plurality of needle electrodes. The interface board may define a plurality of interface board openings, the electrical interface spacer may define a plurality of electrical spacer openings extending within the electrical interface spacer, and the biological interface spacer may define a plurality of biological spacer openings extending within the biological interface spacer. A first portion of each needle electrode of the plurality of needle electrodes may be received in a corresponding opening of the plurality of electrical spacer openings and a second portion of each needle electrode may be received in a corresponding opening of the plurality of biological spacer openings. Each needle electrode of the plurality of needle electrodes extends along a longitudinal axis.

Each needle electrode of the plurality of needle electrodes may include a head portion in the proximal electrical interface region and a tip portion terminating the distal biological interface region and configured to penetrate into tissue. The head portion may include at least three contact surfaces. Each of the at least three contact surfaces may extend along a length parallel to the longitudinal axis. The head portion may also include non-contact surfaces between each of the at least three contact surfaces. Each of the at least three contact surfaces may be configured to contact and electrically couple to a metalized surface of a corresponding interface board opening of the plurality of interface board openings and each of the non-contact surfaces may be configured to lack contact with the metalized surface of the corresponding interface board opening when the head portion is received therein.

In one or more embodiments, the second portion of each needle electrode of the plurality of needle electrodes may also include a biological spacer coupling portion configured to engage a surface defining the corresponding opening of the plurality of biological spacer openings in which it may be received to maintain the needle electrode in a fixed position within the biological interface spacer. In one or more embodiments, each needle electrode of the plurality of needle electrodes may include an upper shaft between the head portion and the biological spacer coupling portion and a lower shaft between the biological spacer coupling portion and the tip portion. The biological spacer coupling portion may include a recessed region proximate the upper shaft and an expanded region between the recessed region and the lower shaft. A diameter of the recessed region may be less than a diameter of the expanded region and a diameter of the upper shaft; and the diameter of the upper shaft may be greater than the diameter of the expanded region.

In one or more embodiments, each needle electrode of the plurality of needle electrodes may extend through a corresponding biological spacer opening of the plurality of biological spacer openings such that the expanded region of the needle electrode may contact the surface defining the corresponding biological spacer opening to provide an interference fit between the needle electrode and the biological interface spacer. In one or more embodiments, the diameter of the upper shaft may be greater than a diameter of the biological spacer opening and the diameter of the upper shaft may restrict the biological interface spacer from moving past the upper shaft towards the electrical interface spacer. In one or more embodiments, each of the plurality of electrical spacer openings may extend from an electrical spacer first surface to an electrical spacer second surface opposing the electrical spacer first surface. The plurality of needle electrodes may extend through corresponding openings of the plurality of electrical spacer openings. In one or more embodiments, the plurality of biological spacer openings may extend from a biological spacer first surface facing the electrical interface spacer to a biological spacer second surface opposing the biological spacer first surface. The plurality of needle electrodes may extend through corresponding openings of the plurality of biological spacer openings.

In one or more embodiments, each contact surface of the at least three contact surfaces may define a contact surface area equal to the contact surface area of each of the other contact surfaces. In one or more embodiments, the at least three contact surfaces may be equally spaced apart about the longitudinal axis. In one or more embodiments, each non-contact surface may extend along a length parallel to the longitudinal axis and each non-contact surface may define an equal width perpendicular to the longitudinal axis between each contact surface of the at least three contact surfaces. In one or more embodiments, the at least three contact surfaces may include four contact surfaces equally spaced apart about the longitudinal axis. In one or more embodiments, the electrical interface spacer may lie in an electrical spacer plane and the biological interface spacer may lie in a biological spacer plane. The longitudinal axis of each needle electrode may be normal to both of the electrical spacer plane and the biological spacer plane. In one or more embodiments, each needle electrode of the plurality of needle electrodes may be positioned less than 2 millimeters from another needle electrode.

Another exemplary needle probe array described herein may include an interface board located at a proximal electrical interface region of the needle probe array, a biological interface spacer located at a distal biological interface region of the needle probe array, and a plurality of needle electrodes. The interface board may define a plurality of interface board openings and the biological interface spacer may define a plurality of biological spacer openings extending within the biological interface spacer. A first portion of each needle electrode of the plurality of needle electrodes may be received in a corresponding opening of the plurality of interface board openings and a second portion of each needle electrode may be received in a corresponding opening of the plurality of biological spacer openings. Each needle electrode of the plurality of needle electrodes extends along a longitudinal axis.

Each needle electrode of the plurality of needle electrodes may include a head portion in the proximal electrical interface region and a tip portion terminating the distal biological interface region and configured to penetrate into tissue. The head portion may include at least three contact surfaces. Each of the at least three contact surfaces may extend along a length parallel to the longitudinal axis. The head portion may also include non-contact surfaces between each of the at least three contact surfaces. Each of the at least three contact surfaces may be configured to contact and electrically couple to a metalized surface of a corresponding interface board opening of the plurality of interface board openings and each of the non-contact surfaces may be configured to lack contact with the metalized surface of the corresponding interface board opening when the head portion is received therein.

In one or more embodiments, the array may also include an electrical interface spacer located at the proximal electrical interface region of the needle probe array. The electrical interface spacer may define a plurality of electrical spacer openings extending within the electrical interface spacer. The first portion of each needle electrode of the plurality of needle electrodes may be received in a corresponding opening of the plurality of electrical spacer openings. The plurality of electrical spacer openings may be aligned with the plurality of interface board openings. In one or more embodiments, each needle electrode of the plurality of needle electrodes may define a head portion end surface and the head portion end surface may be positioned a distance from an interface board first surface. In one or more embodiments, the second portion of each needle electrode of the plurality of needle electrodes may also include a biological spacer coupling portion configured to engage a surface defining the corresponding opening of the plurality of biological spacer openings in which it may be received to maintain the needle electrode in a fixed position within the biological interface spacer.

In one or more embodiments, each needle electrode of the plurality of needle electrodes may include an upper shaft between the head portion and the biological spacer coupling portion and a lower shaft between the biological spacer coupling portion and the tip portion. The biological spacer coupling portion may include a recessed region proximate the upper shaft and an expanded region between the recessed region and the lower shaft. A diameter of the recessed region may be less than a diameter of the expanded region and a diameter of the upper shaft; and the diameter of the upper shaft may be greater than the diameter of the expanded region. Each needle electrode of the plurality of needle electrodes may extend through a corresponding biological spacer opening of the plurality of biological spacer openings such that the expanded region of the needle electrode contacts the surface defining the corresponding biological spacer opening to provide an interference fit between the needle electrode and the biological interface spacer.

An exemplary method of manufacturing a needle probe array described herein may include providing an electrical interface spacer defining a plurality of electrical spacer openings extending from an electrical spacer first surface to an electrical spacer second surface opposing the electrical spacer first surface. The method may also include positioning an interface board adjacent the electrical spacer first surface. The interface board may define a plurality of interface board openings extending from an interface board first surface to an interface board second surface opposing the interface board first surface. The interface board second surface may be positioned facing the electrical spacer first surface and the interface board may be positioned such that the plurality of interface board openings align with the plurality of electrical spacer openings.

The method may further include loading a plurality of needle electrodes into the plurality of interface board openings and then through the plurality of electrical spacer openings. Each needle electrode of the plurality of needle electrodes may include a tip portion configured to penetrate into skin, a head portion, and a biological spacer coupling portion located between the head portion and the tip portion. The tip portion may pass through the plurality of interface board openings and the plurality of electrical spacer openings when loading the plurality of needle electrodes. The head portion may include at least three contact surfaces and may also include non-contact surfaces between each of the at least three contact surfaces. The head portion of each needle electrode may not pass through the plurality of interface board openings when loading the plurality of needle electrodes.

The method may also include applying force to the plurality of needle electrodes relative to the electrical interface spacer and interface board such that the head portions of the plurality of needle electrodes may be moved within the plurality of interface board openings. Each of the at least three contact surfaces of the head portions of each of the plurality of needle electrodes may contact and electrically couple to a metalized surface of a corresponding interface board opening of the plurality of interface board openings and the non-contact surfaces may lack contact with the metalized surface of the interface board opening. The method may further include providing a biological interface spacer defining a plurality of biological spacer openings extending from a biological spacer first surface to a biological spacer second surface opposing the biological spacer first surface and inserting the tip portions of the plurality of needle electrodes through the plurality of biological spacer openings. The method may further yet include applying force to the plurality of needle electrodes relative to the biological interface spacer such that the biological spacer coupling portion of each needle electrode may be fixed at a location within the biological interface spacer. The biological spacer second surface may be positioned a distance from the tip portions of the plurality of needle electrodes.

In one or more embodiments, applying force to the plurality of needle electrodes relative to the electrical interface spacer and interface board may include applying force at the head portion of each needle electrode of the plurality of needle electrodes. The head portion may include a taper region to facilitate movement of the head portion into the plurality of interface board openings when the force may be applied.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is perspective view of an exemplary needle probe array (e.g., including a plurality of needle electrodes) positioned proximate biological tissue (e.g., skin) and received by an exemplary interface board.

FIG. 2 is an exploded side view of the exemplary needle probe array of FIG. 1.

FIG. 3 is a side view of an exemplary needle electrode of the exemplary needle probe array.

FIG. 4A is a perspective view of a head portion of the exemplary needle electrode of FIG. 3.

FIG. 4B is top view of the head portion of the exemplary needle electrode of FIG.

3.

FIG. 4C is bottom view of the tip portion of the exemplary needle electrode of

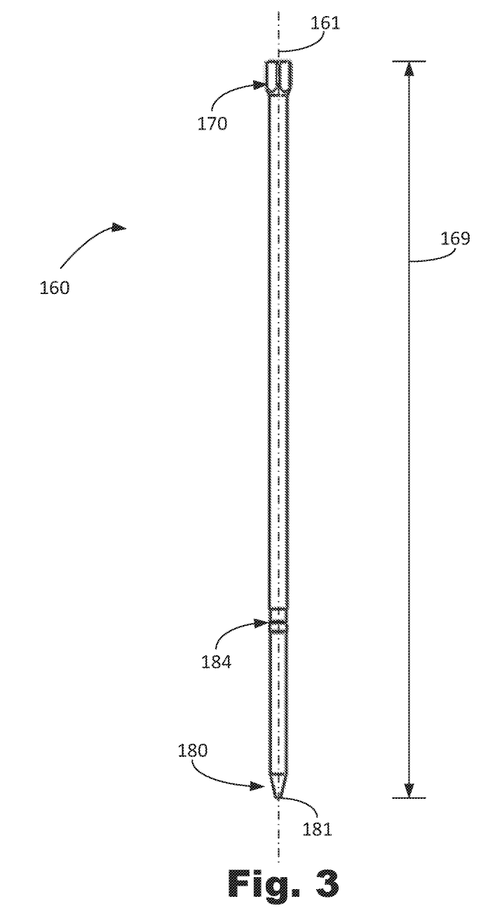

FIG. 3.

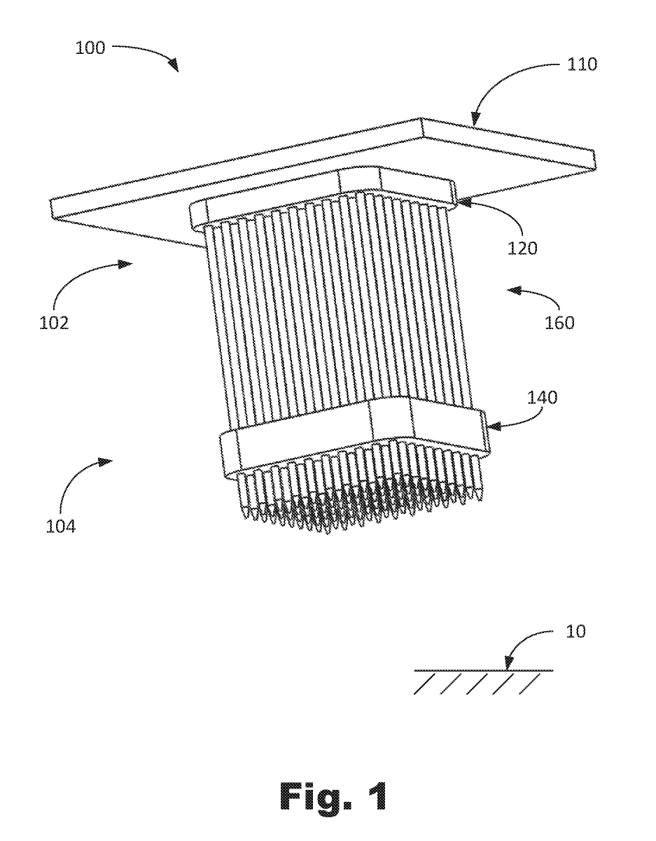

FIG. 5 is a top view of the exemplary plurality of needle electrodes of FIG. 1 received in the exemplary interface board also shown therein.

FIG. 6A is a side perspective view of an exemplary biological spacer coupling portion of the exemplary needle electrode of FIG. 3

FIG. 6B is a side plan view of the exemplary biological spacer coupling portion of the exemplary needle electrode of FIG. 3.

FIG. 7 is a cross section view along a plane parallel and through the center of a row of needle electrodes of the exemplary needle probe array of FIG. 1.

FIG. 8A is an expanded view of a portion of the cross section view of FIG. 7 as identified therein.

FIG. 8B is another expanded view of a portion of the cross section view of FIG. 7 as identified therein.

FIG. 9 is a block diagram of an exemplary method of manufacturing the exemplary needle probe array of FIGS. 1-8.

### DETAILED DESCRIPTION OF EXEMPLARY EMBODIMENTS

In the following detailed description of illustrative embodiments, reference is made to the accompanying figures of the drawing, which form a part hereof, and in which are shown, by way of illustration, specific embodiments which may be practiced. It is to be understood that other embodiments may be utilized and structural changes may be made without departing from (e.g., still falling within) the scope of the disclosure presented hereby.

Exemplary apparatus, systems, structures, and methods shall be described with reference to FIGS. 1-9. It will be apparent to one skilled in the art that elements from one embodiment may be used in combination with elements of the other embodiments, and that the possible embodiments of such apparatus and systems using combinations of features set forth herein is not limited to the specific embodiments shown in the Figures and/or described herein. Further, it will be recognized that the embodiments described herein may include many elements that are not necessarily shown to scale. Still further, it will be recognized that the size and shape of various elements herein may be modified but still fall within the scope of the present disclosure, although certain one or more shapes and/or sizes, or types of elements, may be advantageous over others.

The present disclosure relates generally to a needle probe array that includes a plurality of needle electrodes that may be stabilized for insertion into biological tissue (e.g., skin of a being, human or otherwise). Further, the needle probe array may provide consistent contact with an interface board in which a plurality of needle electrodes thereof are received (e.g., to reduce signal variability).

The plurality of needle electrodes may be electrically coupled to the interface board such that signals (e.g., electric currents) may be transmitted in a like manner between the interface board (and/or a circuit board or system attached thereto) and each needle electrode of the plurality of needle electrodes. Due to the electrical connection between each needle electrode and the interface board, a stimulating signal (e.g., in the form of an electrical current) may be transmitted from the interface board to the plurality of needle electrodes and into the skin and/or a biopotential measurement may be taken within and/or from tissue (e.g., skin) and transmitted through the plurality of needle electrodes to the interface board. The interface board may be electrically coupled to a circuit board or other portions of a system (e.g., a stimulation system configured to stimulate tissue, a diagnostic system for analysis of signals received from tissue, etc.) from which an electrical current may be transmitted to and/or received from the plurality of needle electrodes.

The plurality of needle electrodes may make contact with the interface board within a plurality of interface board openings. Specifically, each needle electrode of the plurality of needle electrodes may make contact with a metalized surface within (e.g., lining) a corresponding interface board opening of the plurality of interface board openings. Each needle electrode may define contact surfaces that are configured to contact the metalized surface of the interface board opening. Further, each needle may define non-contact surfaces between the contact surfaces that lack contact with (or do not contact) the metalized surface of the interface board opening.

These contact surfaces of the needle electrodes may provide a connection with the metalized surface of the interface board opening (e.g., in view of the spacing of such contact surfaces) such that a more stable and consistent electrical connection may be made between the needle electrode and the surface of the interface board opening. For example, the contact surfaces of each needle electrode of the array provide the same distinct and specific surfaces (e.g., of substantially equal area and positioning) that protrude outward from the axis of the needle electrode such that the contact surfaces of each needle electrode of the array contact the metalized surface of the corresponding interface board opening in which they are received in substantially the same manner.

On the other hand, needle electrodes that define entirely conforming shapes that are similar to the shape of the interface board opening (e.g., a cylindrical conforming shape of the needle electrode being receiving in a metalized cylindrical opening of the interface board) with which it contacts may produce inconsistent contact between portions of the needle electrode and the corresponding opening in which it is received (e.g., portions of the conforming shape may not contact the metalized surface of the interface board opening). When the placement and size of this inconsistent contact occurs differently among the needle electrodes of the array, variability problems across the array of needle electrodes occurs with respect to the electrical connection between such needle electrodes and the interface board.

By intentionally defining contact surfaces and non-contact surfaces, the variability of where the needle electrode contacts the surface of the interface board opening and where the needle electrode does not contact the surface of the interface board opening may be controlled and reduced. Controlling this variability may provide a more consistent connection across the needle electrodes of the array and the interface board.

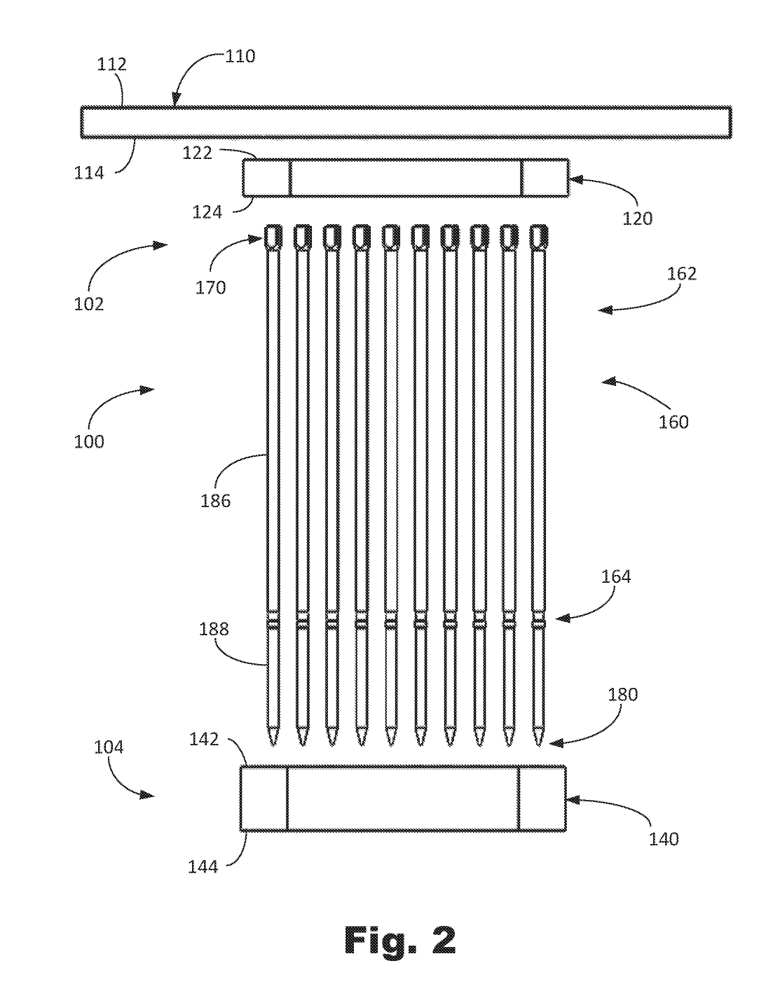

An exemplary needle probe array 100 that may be coupled to an interface board 110 defining a plurality of interface board openings 116 (e.g., as shown in FIG. 8A) is shown in FIGS. 1-2. The needle probe array 100 may be configured to be inserted into tissue (e.g., skin 10) to stimulate (e.g., tissue or cells thereof) using electrical signals or measure electrical signals of, e.g., tissue or cells thereof within a body. The needle probe array 100 may define a proximal electrical interface region 102 and a distal biological interface region 104. The proximal electrical interface region 102 may include components relating to the interface (e.g., providing of electrical signals) between the needle probe array 100 and other components of a system coupled thereto (e.g., a diagnostic or stimulation system including components such as interface boards, circuit boards, wires, processors, power sources, etc.) and the distal biological interface region 104 may include components relating to the interface between the needle probe array 100 and biological tissue (e.g., needle tips, spacers, guards, sterilized components, fluid barriers, etc.). Further, the proximal electrical interface region 102 may be at a location at which force is applied to insert the needle probe array 100 into biological tissue (e.g., through skin 10).

The distal biological interface region 104 may be positioned proximally near the user when the needle probe array 100 is in use. The proximal electrical interface region 102 may be located farther from the user when the needle probe array 100 is in use. The needle probe array 100 is configured as such to separate (e.g., space) the proximal electrical interface region 102, and thus, the electrical components of the needle probe array 100 away from the distal biological interface region 104, and thus, the biological components (e.g., skin, biological fluids, etc.).

The needle probe array 100 may include an electrical interface spacer 120 located at (e.g., within, proximate, near, etc.) the proximal electrical interface region 102 of the needle probe array 100. The electrical interface spacer 120 may comprise various different materials such as, e.g., polymer, plastic, any suitable insulative material, etc. The electrical interface spacer 120 may define an electrical spacer first surface 122 and an electrical spacer second surface 124 opposing the electrical spacer first surface 122. The electrical interface spacer 120 may define a thickness between the electrical spacer first and second surfaces 122, 124. The thickness of the electrical interface spacer 120 may be, e.g., greater than or equal to 0.6 millimeters, greater than or equal to 0.8 millimeters, greater than or equal to 1 millimeter, greater than or equal to 1.2 millimeters, etc. and/or less than or equal to 1.8 millimeters, less than or equal to 1.6 millimeters, less than or equal to 1.4 millimeters, less than or equal to 1.1 millimeters, etc. In one embodiment, the thickness of the electrical interface spacer 120 may be, e.g., greater than or equal to 0.8 millimeters and/or less than or equal to 1.6 millimeters. In one or more embodiments, the electrical interface spacer 120 may provide insulation between electrical components at the electrical interface region 102 and the biological interface region 104. Further, the electrical interface spacer 120 may provide electrical insulation between the plurality of needle electrodes of the needle probe array 100. Also, the electrical interface spacer 120 (e.g., at the electrical spacer first surface 122, at the electrical spacer second surface 124, or therebetween) may be provided in one or more various different shapes (e.g., square, rectangular, circular, triangular, etc.).

The electrical interface spacer 120 may define a plurality of electrical spacer openings 126 (e.g., as shown in FIGS. 7 and 8A) extending within the electrical interface spacer 120. In one or more embodiments, each of the plurality of electrical spacer openings 126 is sized for receiving an electrode needle of the needle probe array 100.

Further, in one or more embodiments, each of the plurality of electrical spacer openings 126 may extend through the electrical interface spacer 120 from the electrical spacer first surface 122 to the electrical spacer second surface 124 for receiving a corresponding electrode needle. However, in one or more other embodiments, various electrical connection structures may be provided in the electrical spacer 120 such that the openings 126 need not extend entirely through the electrical interface spacer 120. For example, in one or more embodiments, a plurality of electrical spacer openings 126 may extend from the electrical spacer first surface 122 and into the electrical interface spacer 120 (e.g., not to the electrical spacer second surface 124) and/or a plurality of electrical spacer openings 126 may extend from the electrical spacer second surface 124 and into the electrical interface spacer 120 (e.g., not to the electrical spacer first surface 122). In such embodiments, electrical connection structures (e.g., electrical feedthroughs) within the electrical interface spacer 120 forming a part of the needle electrodes may be used to connect other portions of the needle electrodes on one or more opposing sides of the electrical interface spacer 120 instead of a single integral needle electrode being provided entirely through the electrical spacer 120.

The needle probe array 100 may also include a biological interface spacer 140 located at (e.g., within, proximate, near, etc.) the distal biological interface region 104 of the needle probe array 100. In one or more embodiments, the biological interface spacer 140 may be described as a “depth guard” (e.g., wherein the spacer 140 limits the depth to which the needle electrodes may be inserted into tissue). The biological interface spacer 140 may define a biological spacer first surface 142 and a biological spacer second surface 144 opposing the biological spacer first surface 142. For example, the biological spacer second surface 144 provides the depth limit to which the needle electrodes may be inserted into tissue (e.g., the surface 144 limits this depth when it comes into contact with tissue).

The biological interface spacer 140 may comprise various different materials such as, e.g., polymer, plastic, any suitable insulative material, etc. The biological interface spacer 140 may define a thickness between the biological spacer first and second surfaces 142, 144. The thickness of the biological interface spacer 140 may be, e.g., greater than or equal to 1.5 millimeters, greater than or equal to 2 millimeters, greater than or equal to 2.25 millimeters, greater than or equal to 2.5 millimeters, etc. and/or less than or equal to 3.5 millimeters, less than or equal to 3 millimeters, less than or equal to 2.75 millimeters, less than or equal to 2.4 millimeters, etc. In one embodiment, the thickness of the biological interface spacer 140 may be, e.g., greater than or equal to 2 millimeters and/or less than or equal to 3 millimeters. In one or more embodiments, the biological interface spacer 140 may provide a separation between biological components at the biological spacer interface region 104 and the electrical interface region 102. Further, the biological interface spacer 140 may provide electrical insulation between the plurality of needle electrodes of the needle probe array 100. Also, the biological interface spacer 140 (e.g., at the biological spacer first surface 142, at the biological spacer second surface 144, or therebetween) may be provided in one or more various different shapes (e.g., square, rectangular, circular, triangular, etc.).

The biological interface spacer 140 may define a plurality of biological spacer openings 146 (e.g., as shown in FIGS. 7 and 8B) extending within the biological interface spacer 140. In one or more embodiments, the plurality of biological spacer openings 146 may extend through the biological interface spacer 140 from the biological spacer first surface 142 to the biological spacer second surface 144 for receiving corresponding needle electrodes. However, in one or more other embodiments, various electrical connection structures may be provided in the biological interface spacer 140 such that the openings 146 need not extend entirely through the biological interface spacer 140. For example, in one or more embodiments, the plurality of biological spacer openings 146 may extend from the biological spacer first surface 142 and into the biological interface spacer 140 (e.g., not to the biological spacer second surface 144) and/or the plurality of biological spacer openings 146 may extend from the biological spacer second surface 144 and into the electrical interface spacer 140 (e.g., not to the biological spacer first surface 142). In such embodiments, electrical connection structures (e.g., electrical feedthroughs) within the biological interface spacer 140 forming a part of the needle electrodes may be used to connect other portions of the needle electrodes on one or more opposing sides of the biological interface spacer 140 instead of a single integral needle electrode being provided entirely through the biological interface spacer 140.

The needle probe array 100 may also include a plurality of needle electrodes 160 extending from the proximal electrical interface region 102 to the distal biological interface region 104. Each needle electrode 160 of the plurality of needle electrodes 160 may extend along a longitudinal axis 161 (e.g., as shown in FIG. 3). In one or more embodiments, the plurality of needle electrodes 160 may extend parallel to one another. Each needle electrode 160 of the plurality of needle electrodes may include a first portion 162 along the axis 161 that is received in a corresponding opening of the plurality of electrical spacer openings 126 and may define a second portion 164 along the axis 161 that is received in a corresponding opening of the plurality of biological spacer openings 146.

In one or more embodiments, the needle probe array 100 may further include an interface board 110 located at (e.g., within, proximate, near, etc.) the proximal electrical interface region 102 of the needle probe array 100. The interface board 110 may comprise various different materials such as, e.g., polymer, metalized polymer, etc. The interface board 110 may define an interface board first surface 112 and an interface board second surface 114 opposing the interface board first surface 112. The interface board 110 may define a thickness between the interface board first and second surfaces 112, 114. The thickness of the interface board 110 may be, e.g., greater than or equal to 1 millimeter, greater than or equal to 1.25 millimeters, greater than or equal to 1.5 millimeters, etc. and/or less than or equal to 2 millimeters, less than or equal to 1.8 millimeters, less than or equal to 1.7 millimeters, etc. In one embodiment, the thickness of the interface board 110 may be, e.g., greater than or equal to 1.5 millimeters and/or less than or equal to 1.7 millimeters. Also, the interface board 110 (e.g., at the interface board first surface 112, at the interface board second surface 114, or therebetween) may be provided in one or more various different shapes (e.g., square, rectangular, circular, triangular, etc.).

The interface board 110 may define a plurality of interface board openings 116 (e.g., as shown in FIG. 7 and FIG. 8A) extending within the interface board 110. In one or more embodiments, the plurality of interface board openings 116 may extend through the interface board 110 from the interface board first surface 112 to the interface board second surface 114. However, in one or more other embodiments, the plurality of interface board openings 116 may extend from the interface board second surface 114 and into the interface board 110 (e.g., not to the interface board first surface 112). Each of the plurality of needle electrodes 160 extend through at least a portion of a corresponding opening of the plurality of interface board openings 116 when received therein.

In one or more embodiments, the interface board 110 may be any board including metallized openings suitable for receiving a portion of the needle electrodes 160 therein. For example, interface board 110 may be a circuit board including a plurality of metallized openings therein (e.g., such as metallized vias through the circuit board; such metallized vias providing connections to various external pads through one or more interconnect layers or traces). In other embodiments, the interface board 110 may be electrically and/or physically connected to a circuit board via one or more electrical and/or mechanical connection structures. The plurality of needle electrodes 160 may extend through at least a portion of the plurality of interface board openings 116 and be configured to provide an electrical connection from the circuit board to each needle electrode 160 of the plurality of needle electrodes 160. For example, the circuit board may be configured to transmit electrical signals to the plurality of needle electrodes 160 and/or receive electrical signals from the plurality of needle electrodes 160.

In one or more embodiments, a control apparatus or controller (e.g., one or more processors employing one or more programs or routines carrying out one or more methods or processes and implemented with one or more types of memory) may be configured to control the device and/or one or more elements thereof (e.g., transmitting electrical signals, measuring electrical signals, etc.) through the circuit board (e.g., the interface board 110). In one or more embodiments, the control apparatus may be configured to perform stimulation routines, diagnostic routines, or the like relating to signals transmitted to and/or received from the needle probe array 100.

The methods and/or logic and/or configurations described in this disclosure, including those attributed to the devices, or various constituent components, may be implemented, at least in part, in hardware, software, firmware, or any combination thereof. For example, various aspects of the techniques may be implemented within one or more processors, including one or more microprocessors, microcontrollers, DSPs, ASICs, FPGAs, or any other equivalent integrated or discrete logic circuitry, as well as any combinations of such components, or other devices. The term “processor” or “processing circuitry” may generally refer to any of the foregoing logic circuitry, alone or in combination with other logic circuitry, or any other equivalent circuitry.

The plurality of needle electrodes 160 may be arranged in the interface board 110 a multitude of different ways. For example, the plurality of needle electrodes 160 may be arranged in the plurality of interface board openings 116 in a grid or matrix. As shown in FIG. 5, the plurality of needle electrodes 160 are arranged in a rectangular grid that is three rows of needle electrodes 160 by four columns of needle electrodes 160. The grid may include any number of columns and rows of needle electrodes 160. For example, the grid may include less than or equal to 50 rows of needle electrodes 160 and less than or equal to 50 columns of needle electrodes 160. Further, for example, the grid may include more than or equal to 3 rows of needle electrodes 160 and more than or equal to 3 columns of needle electrodes 160. In one or more embodiments, the plurality of needle electrodes 160 may be arranged in other configurations such as, e.g., a square array of electrodes, multiple concentric circular rings of electrodes, a triangular arrangement of electrodes, an offset arrangement of electrodes, etc.

Further, in one or more embodiments, the cross-section of the needle electrodes 160 (e.g., such as head portion 170) orthogonal to axis 161, as well as the cross-section of the corresponding interface board openings 116 in which they are received may be provided in one or more various shapes such as, e.g., circular, square, triangular, hexagonal, etc. As shown in FIG. 5, each needle electrode 160 defines a square cross-sectional shape with rounded corners. The rounded corners may define a contact surface 172 at which the needle electrode 160 contacts a surface of the interface board openings 116 (e.g., circular cross-sectional openings) and non-contact surfaces 174 that lack contact with the surface of the interface board openings 116. The various shapes of needle electrodes may include shaped portions that define contact surfaces which conform to one or more portions of a shape of the interface board openings 116. In one or more embodiments, each needle electrode 160 of the plurality of needle electrodes 160 may be positioned less than or equal to 2 millimeters from another needle electrode 160, less than or equal to 1.27 millimeters from another needle electrode 160, less than or equal to 1 millimeter from another needle electrode 160, or less than or equal to 0.8 millimeters from another needle electrode 160.

Further with reference to the exemplary needle electrode 160 of the plurality of needle electrodes 160 shown in FIG. 3, each needle electrode 160 of the plurality of needle electrodes 160 may include a head portion 170, a tip portion 180, and a biological spacer coupling portion 184 between the head portion 170 and the tip portion 180. Each needle electrode 160 may extend along the longitudinal axis 161 between the head portion 170 and the tip portion 180. The head portion 170 of each needle electrode 160 may be in the proximal electrical interface region 102. The tip portion 180 may terminate the distal biological interface region 104 and may be configured to penetrate into tissue (e.g., skin 10). Each needle electrode 160 may define a length 169 along the longitudinal axis 161 from the head portion 170 to the tip portion 180 that is, e.g., greater than or equal to 10 millimeters, greater than or equal to 15 millimeters, greater than or equal to 20 millimeters, greater than or equal to 25 millimeters, etc. and/or less than or equal to 50 millimeters, less than or equal to 40 millimeters, less than or equal to 30 millimeters, less than or equal 22 millimeters, etc.

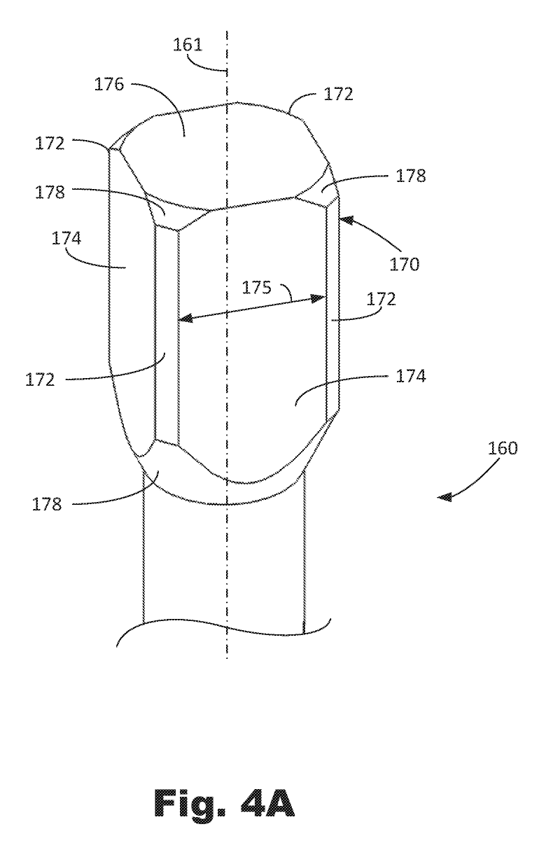

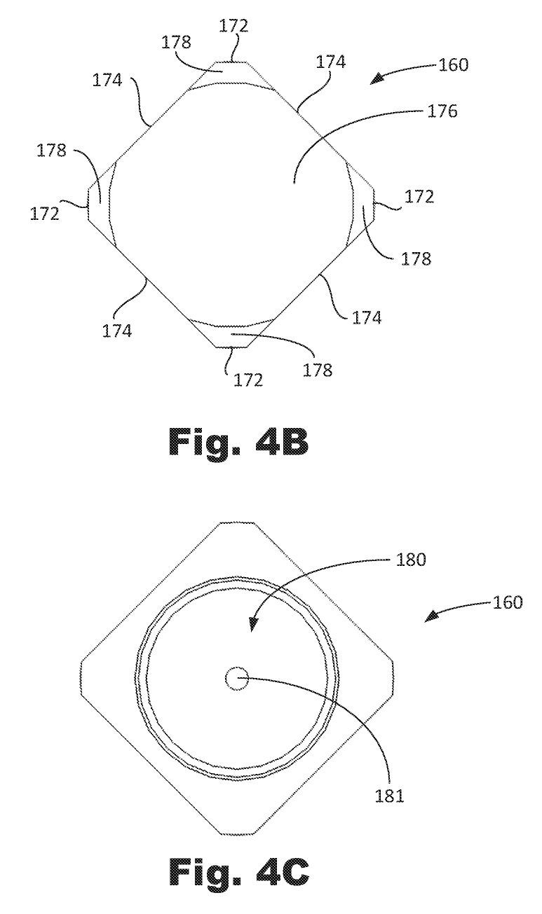

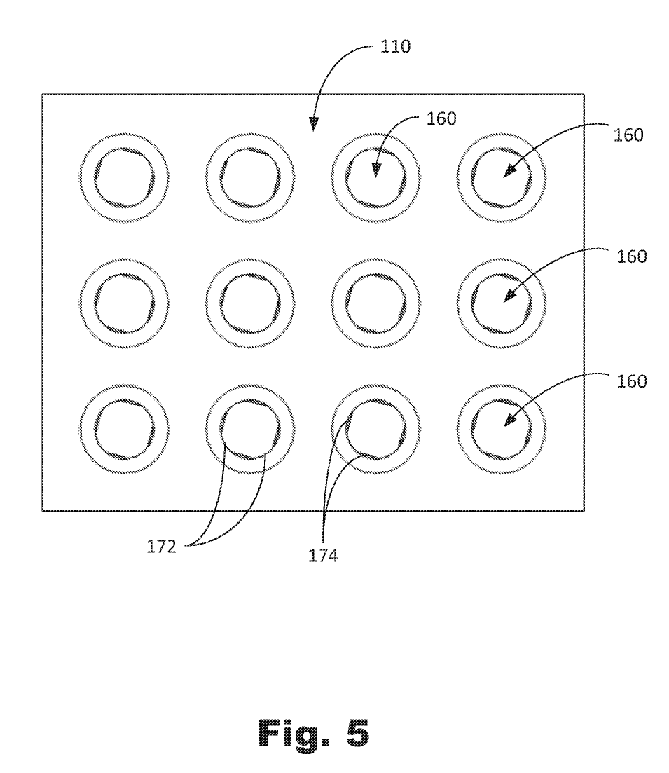

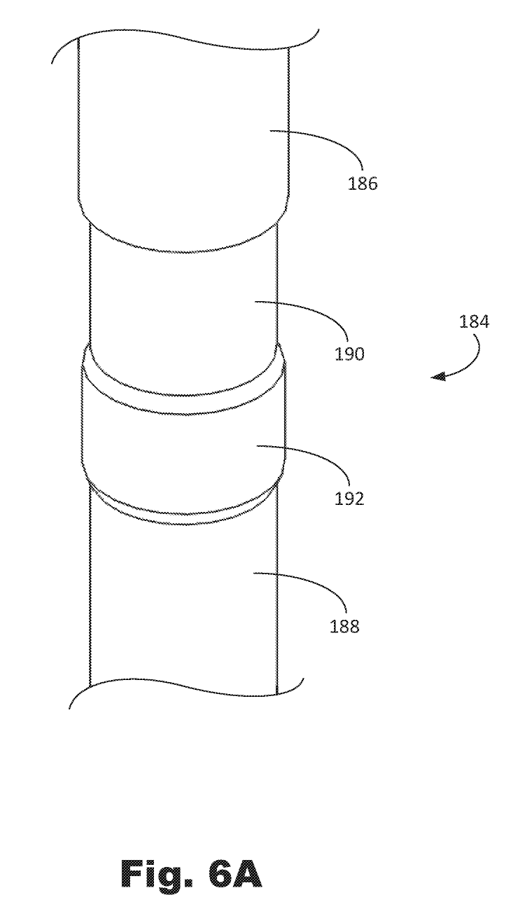

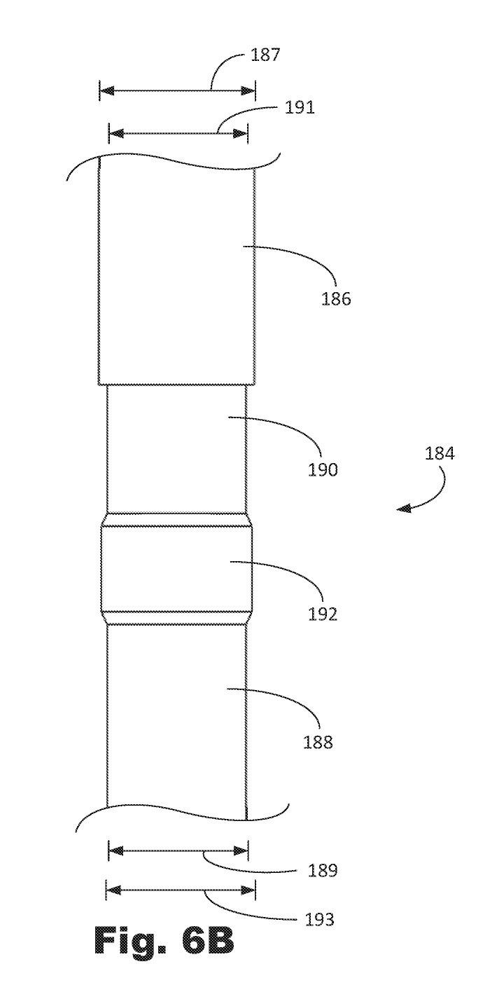

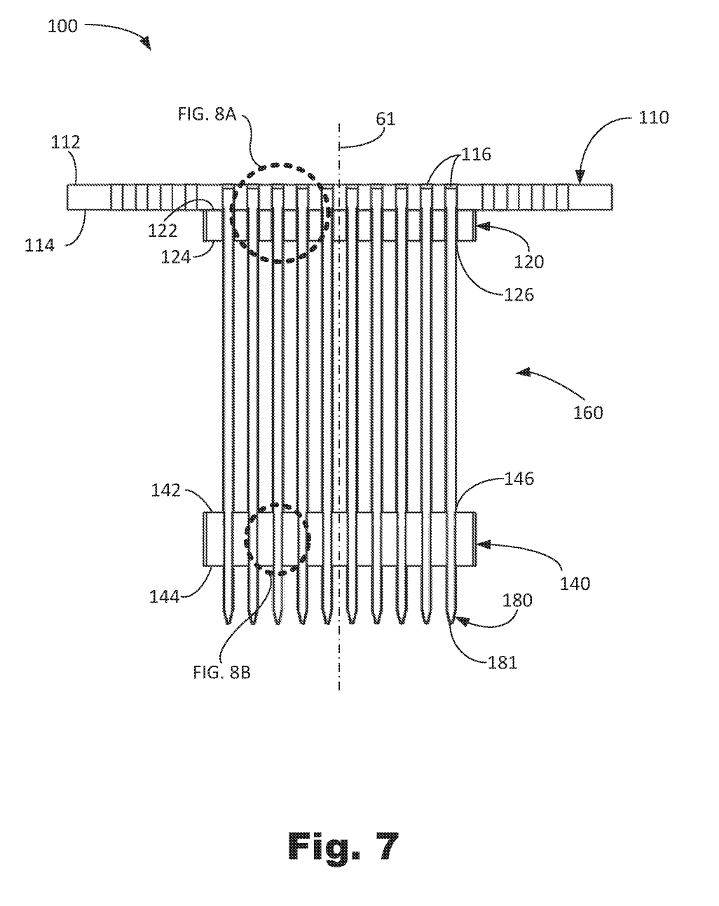

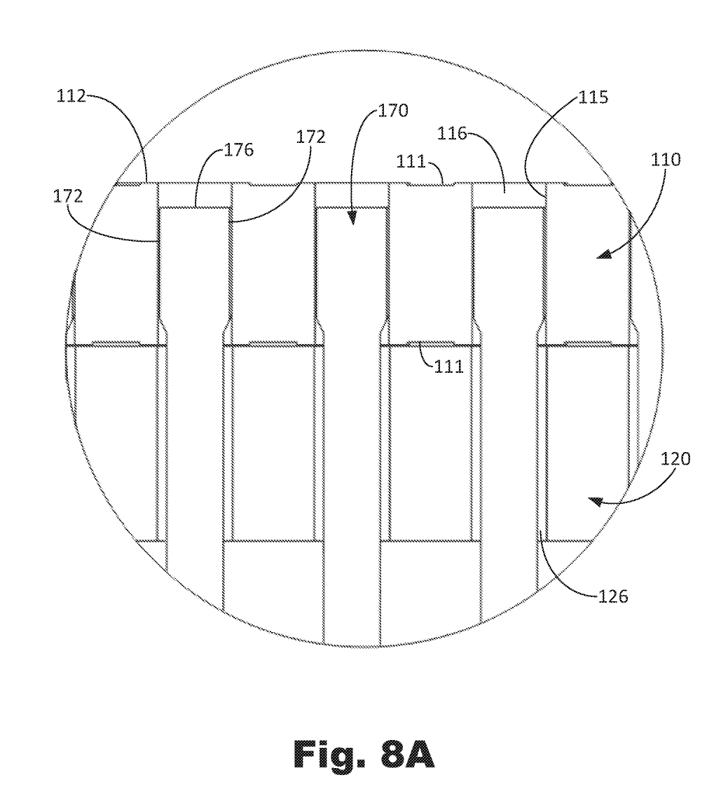

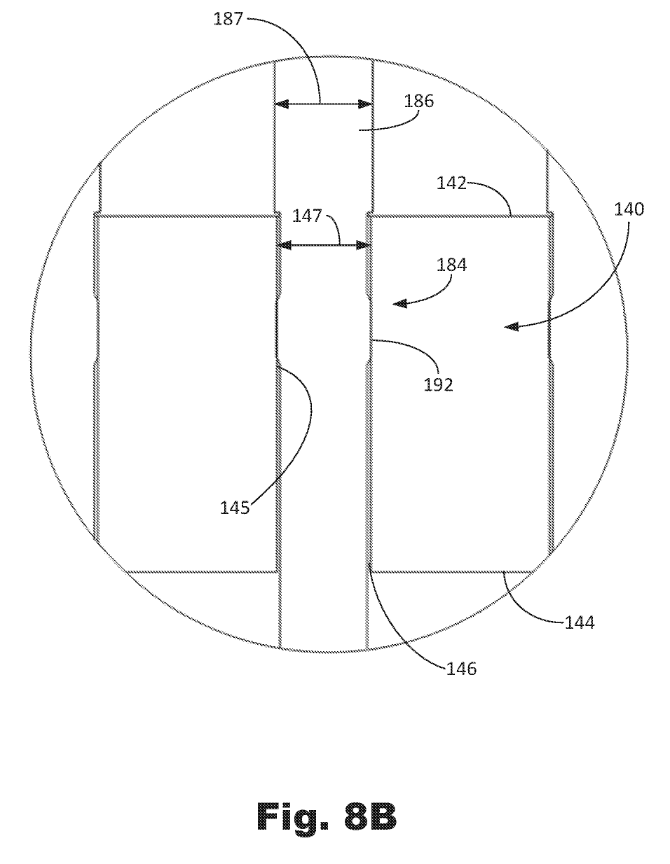

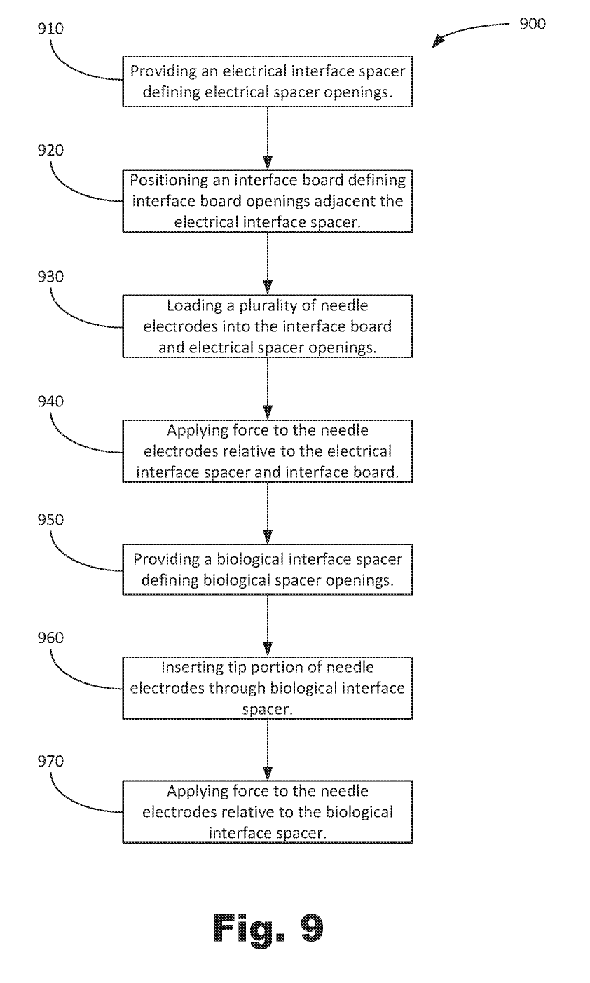

An enlarged view of a head portion 170 of one needle electrode 160 of the plurality of needle electrodes 160 is shown in FIG. 4A. In one or more embodiments, the head portion 170 of each needle electrode 160 may include at least three contact surfaces 172. Each of the at least three contact surfaces 172 may be configured to contact and electrically couple to a surface 115 (e.g., as shown in FIG. 8A) of a corresponding interface board opening 116 of the plurality of interface board openings 116 when the head portion 170 is received therein. In one or more embodiments, the surface 115 of the interface board opening 116 may be a metalized surface that is configured to conduct electricity between the needle electrode 160 and the interface board 110. The at least three contact surfaces 172 may include any number of contact surfaces 172, e.g., three, four, five, six, etc. However, in at least one embodiment less than five contact surfaces are used. As shown in FIGS. 4A and 4B, the head portion 170 includes four contact surfaces 172.

In at least one embodiment, the configuration of each of the head portions 170 of each of the plurality of needle electrodes 160 is the same (e.g., provides the same contact surfaces 172 for contact with the conductive surfaces of corresponding interface board openings 116). Such a configuration reduces the variability in signal from needle electrode to needle electrode when operating under substantially the same conditions.

The contact surfaces 172 of the head portion 170 may be provided in one or more various shapes, sizes, and orientations. For example, as shown in FIG. 4A, the contact surfaces 172 may extend along a length parallel to the longitudinal axis 161. The contact surfaces 172 may extend along the longitudinal axis 161 for about, e.g., greater than or equal to 1.5 millimeters and/or less than or equal to 1.7 millimeters. In one or more embodiments, the contact surfaces 172 may extend along the longitudinal axis 161 for a length less than or equal to a length of the interface board opening 116. The contact surfaces 172 may be various shapes such as, e.g., oblong, rectangular, etc. In one or more embodiments, the contact surfaces 172 may be described as defining a surface that conforms to the surface 115 of the interface board opening 116 (e.g., a rounded surface corresponding to a rounded portion of an interface board opening 116). In one or more embodiments, each contact surface 172 may define a contact surface area equal to the contact surface area of each of the other contact surfaces 172. In other embodiments, the contact surface area may be different between at least two contact surfaces 172 of the multiple contact surfaces 172. Still further, at least in one embodiment, the total contact surface area of all the contact surfaces 172 of each head portion of each needle electrode 160 are substantially equivalent. In other words, at least in one embodiment, each head portion of each needle electrode 160 includes the same total contact surface area for contact with conductive surface of the corresponding interface board opening 116.

The head portion 170 of each needle electrode 160 may also include non-contact surfaces 174 between each of the at least three contact surfaces 172. Each of the non-contact surfaces 174 may be configured to lack contact with the surface 115 of the corresponding interface board opening 116 of the plurality of interface board openings 116 when the head portion 170 is received therein. In one or more embodiments, it may be described that the non-contact surfaces 174 are spaced away from or do not contact the surface 115 of the interface board opening 116.

The non-contact surfaces 174 may be positioned between the at least three contact surfaces 172 to space apart the at least three contact surfaces such that, e.g., the at least three contact surfaces 172 create distinct surfaces that contact the surface 115 of the corresponding interface board opening 116. In one or more embodiments, the at least three contact surfaces 172 are equally spaced apart about the longitudinal axis 161 of the needle electrode 160 (e.g., spaced apart or separated from each other by non-contact surfaces 174). The non-contact surfaces 174 may be various shapes such as, e.g., oblong, rectangular, etc., or any other shape separating the contact surfaces 172. As shown, the non-contact surfaces 174 may extend along a length parallel to the longitudinal axis 161. In one or more embodiments, each of the non-contact surfaces 174 may define an equal width 175 perpendicular to the longitudinal axis 161 between each contact surface 172 of the at least three contact surfaces 172 (e.g., equally spacing each contact surface 172 of the at least three contact surfaces 172 apart).

The head portion 170 may further include a head portion end surface 176 as shown in FIGS. 4A and 4B. The head portion end surface 176 may be located at or terminate the end of the head portion 170 and may be positioned adjacent each of the side surfaces (e.g., the at least three contact surfaces 172 and the non-contact surfaces 174). The head portion 170 may include tapers 178 positioned between the head portion 170 and at least one of the contact surfaces 172 and the non-contact surfaces 174. The head portion 170 may also include tapers 178 positioned adjacent at least one of the contact surfaces 172 and the non-contact surfaces 174 and opposite the head portion end surface 176 (e.g., towards the tip portion 180). The tapers 178 may assist in maneuvering or guiding each needle electrode 160 through a corresponding opening (e.g., of the plurality of interface board openings 116 or of the plurality of electrical spacer openings 126).

The tip portion 180 of a needle electrode 160 is shown in FIG. 4C. The tip portion 180 may include a tip 181 that is configured to taper to a point such that the tip portion 180 may be inserted into tissue. In one or more embodiments, the tip portion 180 may be configured to penetrate the skin to a depth that may be limited to the distance measured between the tip 181 and the biological interface spacer 140 (e.g., the biological spacer second surface 144).

The biological spacer coupling portion 184 of one needle electrode 160 is shown in FIGS. 6A and 6B. The biological spacer coupling portion 184 may be located at the second portion 164 of each needle electrode 160 of the plurality of needle electrodes 160. The biological spacer coupling portion 184 may be configured to engage a surface 145 (shown in FIG. 8B) defining a corresponding opening of the plurality of biological spacer openings 146 in which the biological spacer coupling portion 184 is received. The biological spacer coupling portion 184 may engage with the corresponding opening of the plurality of biological spacer openings 146 to maintain the needle electrode 160 in a fixed position within the biological interface spacer 140 (e.g., fixed relative to the biological interface spacer 140 and relative to the other needle electrodes 160).

Each needle electrode 160 of the plurality of needle electrodes 160 may include an upper shaft 186 between the head portion 170 and the biological spacer coupling portion 184 and a lower shaft 188 between the biological spacer coupling portion 184 and the tip portion 180. The biological spacer coupling portion 184 may include a recessed region 190 proximate the upper shaft 186 and an expanded region 192 between the recessed region 190 and the lower shaft 188. In one or more embodiments, a diameter 191 of the recessed region 190 may be less than a diameter 193 of the expanded region 192 and a diameter 187 of the upper shaft 186. Further, in one or more embodiments, the diameter 187 of the upper shaft 186 may be greater than the diameter 193 of the expanded region 192. The variation of the diameters of the upper shaft 186, the lower shaft 188, the recessed region 190, and the expanded region 192 may assist in allowing the needle electrode 160 to be moved into the biological interface spacer 140 and then be fixed therein in its position relative to the biological interface spacer 140 and the other needle electrodes 160 fixed therein.

For example, the upper shaft 186 may define a diameter 187 of between about 0.40 millimeters and about 0.50 millimeters, and in one embodiment, about 0.457 millimeters. The lower shaft 188 may define a diameter 189 of between about 0.35 millimeters and about 0.45 millimeters, and in one embodiment about 0.406 millimeters. The recessed region 190 may define a diameter 191 of between about 0.35 millimeters and about 0.45 millimeters, and in one embodiment, about 0.406 millimeters. The expanded region 192 may define a diameter 193 of between about 0.39 millimeters and about 0.49 millimeters, and in one embodiment, about 0.442 millimeters.

A cross-section of the needle probe array 100 (e.g., the probe array 100 lying along axis 61) including a plurality of needle electrodes 160 is shown in FIG. 7. As shown, the interface board 110 may be adjacent the electrical interface spacer 120 such that the plurality of interface board openings 116 may be aligned with the plurality of electrical spacer openings 126 and the interface board second surface 114 may be facing the electrical spacer first surface 122. In one or more embodiments, the interface board 110 may be attached to the electrical interface spacer 120 using, e.g., adhesive, laser cut double sided tape, etc. In one or more embodiments, the interface board 110 and the electrical interface spacer 120 may be coupled to one another using at least one needle electrode 160 of the plurality of needle electrodes 160, e.g., by interference fit between the needle electrode 160 and each of the interface board 110 and the electrical interface spacer 120. In one or more embodiments, a diameter of the plurality of electrical spacer openings 126 may be larger than a diameter of the plurality of interface board openings 116 such that, e.g., the plurality of needle electrodes 160 may contact the interference board 110 within the plurality of interference board openings 116 but lack contact with the electrical interface spacer 120 within the plurality of electrical spacer openings 126. Also, as shown in FIG. 7, the electrical spacer second surface 124 may be facing the biological spacer first surface 142 and the plurality of electrical spacer openings 126 may be aligned with the plurality of biological spacer openings 146.

In one or more embodiments, the electrical interface spacer 120 may be separated from the biological interface spacer 140 (e.g., measured between the electrical spacer second surface 124 and the biological spacer first surface 142) by about, e.g., greater than or equal to 5 millimeters, greater than or equal to 10 millimeters, greater than or equal to 15 millimeters, greater than or equal to 20 millimeters, etc. and/or less than or equal to 30 millimeters, less than or equal to 25 millimeters, less than or equal to 22 millimeters, less than or equal 17 millimeters, etc. Also, in one or more embodiments, the biological interface spacer 140 may be separated from the tip 181 of the tip portion 180 (e.g., measured between the biological spacer second surface 144 and the tip 181 of the tip portion 180) by about, e.g., greater than or equal to 1 millimeter, greater than or equal to 2 millimeters, greater than or equal to 4 millimeters, greater than or equal to 6 millimeters, etc. and/or less than or equal 10 millimeters, less than or equal to 8 millimeters, less than or equal to 5 millimeters, less than or equal to 3.5 millimeters, etc.

In one or more embodiments, the electrical interface spacer 120 may be positioned parallel to the biological interface spacer 140 (e.g., all of the electrical spacer first and second surfaces 122, 124 and the biological spacer first and second surfaces 142, 144 being parallel). Further, in one or more embodiments, the electrical interface spacer 120 may lie in an electrical spacer plane and the biological interface spacer 140 may lie in a biological spacer plane. In one or more embodiments, the longitudinal axis 161 of each needle electrode 160 may be normal to at least one of or both of the electrical spacer plane or the biological spacer plane.

The head portion 170 of each needle electrode 160 positioned in a corresponding interface board opening 116 is shown in FIG. 8A (which is an enlarged portion of the cross-sectional view of FIG. 7). The head portion 170 is positioned such that the contact surfaces 172 are electrically contacting and coupled to the surface 115 (e.g., metalized surface) of the corresponding interface board opening 116. Each of the plurality of interface board openings 116 may define a cross-sectional diameter orthogonal to axis 161 of, e.g., greater than or equal to about 0.4 millimeters, greater than or equal to about 0.6 millimeters, greater than or equal to about 0.8 millimeters, etc. and/or less than or equal to about 0.9 millimeters, less than or equal to about 0.7 millimeters, less than or equal to about 0.5 millimeters, etc. Each head portion 170 of the needle electrode 160 may be sized such that an interference fit is created when each head portion 170 of the needle electrode 160 is positioned within the corresponding interface board opening 116.

As shown in FIG. 8A, the head portion end surface 176 may be positioned a distance from the interface board first surface 112. In one or more embodiments, the head portion end surface 176 may be in the same plane as the interface board first surface 112. Additionally, the interface board 110 may include indents 111 in each of the interface board first surface 112 and the interface board second surface 114. In one or more embodiments, portions of the interface board 110 that are not indented 111 may include metal, while the indents 111 may include bare board that may be, e.g., insulative material.

The biological spacer coupling portion 184 of each needle electrode 160 positioned in a corresponding biological spacer opening 146 is shown in FIG. 8B (which is an enlarged portion of the cross-sectional view of FIG. 7). In one or more embodiments, each needle electrode 160 of the plurality of needle electrodes 160 extends through a corresponding biological spacer opening 146 of the plurality of biological spacer openings 146 (e.g., between the biological spacer first surface 142 and the biological spacer second surface 144).

In one or more embodiments, for example, as shown in FIG. 8B, when each needle electrode 160 extends through a corresponding biological spacer opening 146, the expanded region 192 of the biological spacer coupling portion 184 may provide an interference fit between the needle electrode 160 and the biological interface spacer 140. For example, the surface 145 defining the corresponding biological spacer opening 146 may be in contact with the expanded region 192 to provide the interference fit. The biological spacer openings 146 may define a constant diameter 147 from the biological spacer first surface 142 to the biological spacer second surface 144. The expanded region 192 may be slightly larger (e.g., by 0.02 millimeters, by 0.05 millimeters, by 0.1 millimeters, by 0.15 millimeters, etc.) than the corresponding biological spacer opening 146 such that the expanded region 192 contacts and forms an interference fit with the surface 145 defining the corresponding biological spacer opening 146.

Furthermore, in one or more embodiments, the diameter 187 of the upper shaft 186 may be greater than a diameter 147 of the corresponding biological spacer opening 146. With the upper shaft diameter 187 greater than the biological spacer opening diameter 147, the diameter 187 of the upper shaft 186 may restrict the biological interface spacer 140 from moving past the upper shaft 186 and towards the electrical interface spacer 120. In other words, the upper shaft 186 may be described as “catching” on the biological spacer first surface 142 because the upper shaft 186 is too large to fit through the biological spacer opening 146.

One exemplary method 900 of manufacturing a needle probe array is illustrated in FIG. 9. The method 900 may include providing an electrical interface spacer (e.g., electrical interface spacer 120) defining a plurality of electrical spacer openings (e.g., electrical spacer openings 126) extending from an electrical spacer first surface (e.g., electrical spacer first surface 122) to an electrical spacer second surface (e.g., electrical spacer second surface 124) opposing the electrical spacer first surface (block 910). In one or more embodiments, the electrical interface spacer may be placed on a first jig to properly position the electrical interface spacer. The method 900 may also include positioning an interface board (e.g., interface board 110) adjacent the electrical spacer first surface (block 920). The interface board may define a plurality of interface board openings (e.g., interface board openings 116) extending from an interface board first surface (e.g., interface board first surface 112) to an interface board second surface (e.g., interface board second surface 114) opposing the interface board first surface. The interface board second surface may be positioned facing the electrical spacer first surface and the interface board may be positioned such that the plurality of interface board openings align with the plurality of electrical spacer openings. In one or more embodiments, the electrical interface spacer 120 and the interface board 110 may be a single piece, or may be coupled or otherwise formed together, but still provide the openings for receiving the head portions 170 of the needle electrodes 160.

The method 900 may also include loading a plurality of needle electrodes (e.g., plurality of needle electrodes 160) into the plurality of interface board openings (e.g., the needle electrodes may be loaded from the interface board first surface) and then through the plurality of electrical spacer openings (block 930). In one or more embodiments, the needle electrodes may also move through the first jig. Each needle electrode of the plurality of needle electrodes may include a tip portion (e.g., tip portion 180) configured to penetrate into skin, a head portion (e.g., head portion 170), and a biological spacer coupling portion (e.g., biological spacer coupling portion 184) located between the head portion and the tip portion. The tip portion may pass through the plurality of interface board openings and the plurality of electrical spacer openings when loading the plurality of needle electrodes. The head portion may include at least three contact surfaces (e.g., at least three contact surfaces 172) and non-contact surfaces (e.g., non-contact surfaces 174) between each of the at least three contact surfaces.

In one or more embodiments, the head portion of each needle electrode does not pass through (e.g., does not pass entirely through) the plurality of interface board openings when loading the plurality of needle electrodes (block 930). For example, the head portion may not pass through the plurality of interface board openings because of the shape and/or size of the head portion relative to the interface board openings (e.g., the head portion may be larger in cross-section than the cross-section of the interface board openings).

The method 900 may further include applying force to the plurality of needle electrodes relative to the electrical interface spacer and interface board such that the head portions of the plurality of needle electrodes may be moved within the plurality of interface board openings (block 940). In one or more embodiments, the head portions of the needle electrodes may be forced within the interface board and possibly the electrical interface spacer, but not into the first jig.

Each of the at least three contact surfaces of the head portions of each of the plurality of needle electrodes may contact and electrically couple to a metalized surface of a corresponding interface board opening of the plurality of interface board openings (e.g., by an interference fit) and the non-contact surfaces may lack contact with the metalized surface of the interface board opening. In one or more embodiments, the first jig may be removed from the plurality of needle electrodes after the head portions of the needle electrodes are fixed within the interface board. The method 900 may also include providing a biological interface spacer (e.g., biological interface spacer 140) defining a plurality of biological spacer openings (e.g., biological spacer openings 146) extending from a biological spacer first surface (e.g. biological spacer first surface 142) to a biological spacer second surface (e.g., biological spacer second surface 144) opposing the biological spacer first surface (block 950). In one or more embodiments, the biological interface spacer may be provided on a second jig.

The method 900 may also include inserting the tip portions of the plurality of needle electrodes through the plurality of biological spacer openings (block 960). The method 900 may further include applying force to the plurality of needle electrodes relative to the biological interface spacer such that the biological spacer coupling portion of each needle electrode may be fixed (e.g., by an interference fit) at a location within the biological interface spacer (block 970). The biological interface spacer (e.g., measured from the biological spacer second surface) may be positioned a distance from the tip portions of the plurality of needle electrodes. In one or more embodiments, after the biological interface spacer is positioned the second jig may be removed. In one or more embodiments, the distances between electrical interface spacer and the biological interface spacer, as well as the distances between the tip portion and the biological interface spacer, may be verified.

In one or more embodiments, applying force (block 940) to the plurality of needle electrodes relative to the electrical interface spacer and interface board may include applying force at the head portion of each needle electrode of the plurality of needle electrodes. In one or more embodiments, the head portion may include a taper region (e.g., taper regions 178) to facilitate movement of the head portion into the plurality of interface board openings when the force is applied.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The use of numerical ranges by endpoints includes all numbers within that range (e.g. 1 to 5 includes 1, 1.5, 2, 2.75, 3, 3.80, 4, and 5) and any range within that range.

Particular materials and dimensions thereof recited in the disclosed examples, as well as other conditions and details, should not be construed to unduly limit this disclosure. Although the subject matter has been described in language specific to structural features and/or methodological acts, it is to be understood that the subject matter defined in the appended claims is not necessarily limited to the specific features or acts described above. Rather, the specific features and acts described above are disclosed as representative forms of implementing the claims.

What is claimed is:

1. A needle probe array to be coupled to an interface board defining a plurality of interface board openings, wherein the needle probe array comprises:
an electrical interface spacer located at a proximal electrical interface region of the needle probe array, wherein the electrical interface spacer defines a plurality of electrical spacer openings extending within the electrical interface spacer;
a biological interface spacer located at a distal biological interface region of the needle probe array, wherein the biological interface spacer defines a plurality of biological spacer openings extending within the biological interface spacer; and
a plurality of needle electrodes, wherein a first portion of each needle electrode of the plurality of needle electrodes is received in a corresponding opening of the plurality of electrical spacer openings and a second portion of each needle electrode is received in a corresponding opening of the plurality of biological spacer openings, wherein each needle electrode of the plurality of needle electrodes extends along a longitudinal axis and comprises:
a head portion in the proximal electrical interface region comprising at least three contact surfaces, wherein each of the at least three contact surfaces extends along a length parallel to the longitudinal axis, wherein the head portion further comprises non-contact surfaces between each of the at least three contact surfaces, wherein each of the at least three contact surfaces are configured to contact and electrically couple to a metalized surface of a corresponding interface board opening of the plurality of interface board openings and each of the non-contact surfaces are configured to lack contact with the metalized surface of the corresponding interface board opening when the head portion is received therein, and
a tip portion terminating the distal biological interface region and configured to penetrate into tissue.

2. The array of claim 1, wherein the second portion of each needle electrode of the plurality of needle electrodes further comprises a biological spacer coupling portion configured to engage a surface defining the corresponding opening of the plurality of biological spacer openings in which it is received to maintain the needle electrode in a fixed position within the biological interface spacer.

3. The array of claim 2, wherein each needle electrode of the plurality of needle electrodes comprises an upper shaft between the head portion and the biological spacer coupling portion and a lower shaft between the biological spacer coupling portion and the tip portion, wherein the biological spacer coupling portion comprises a recessed region proximate the upper shaft and an expanded region between the recessed region and the lower shaft, wherein a diameter of the recessed region is less than a diameter of the expanded region and a diameter of the upper shaft, and wherein the diameter of the upper shaft is greater than the diameter of the expanded region.

4. The array of claim 3, wherein each needle electrode of the plurality of needle electrodes extends through a corresponding biological spacer opening of the plurality of biological spacer openings such that the expanded region of the needle electrode contacts the surface defining the corresponding biological spacer opening to provide an interference fit between the needle electrode and the biological interface spacer.

5. The array of claim 3, wherein the diameter of the upper shaft is greater than a diameter of the biological spacer opening, wherein the diameter of the upper shaft restricts the biological interface spacer from moving past the upper shaft towards the electrical interface spacer.

6. The array of claim 1, wherein each of the plurality of electrical spacer openings extends from an electrical spacer first surface to an electrical spacer second surface opposing the electrical spacer first surface, and further wherein the plurality of needle electrodes extend through corresponding openings of the plurality of electrical spacer openings.

7. The array of claim 1, wherein the plurality of biological spacer openings extend from a biological spacer first surface facing the electrical interface spacer to a biological spacer second surface opposing the biological spacer first surface, wherein the plurality of needle electrodes extend through corresponding openings of the plurality of biological spacer openings.

8. The array of claim 1, wherein each contact surface of the at least three contact surfaces defines a contact surface area equal to the contact surface area of each of the other contact surfaces.

9. The array of claim 1, wherein the at least three contact surfaces are equally spaced apart about the longitudinal axis.

10. The array of claim 1, wherein each non-contact surface extends along a length parallel to the longitudinal axis, and further wherein each non-contact surface defines an equal width perpendicular to the longitudinal axis between each contact surface of the at least three contact surfaces.

11. The array of claim 1, wherein the at least three contact surfaces comprises four contact surfaces equally spaced apart about the longitudinal axis.

12. The array of claim 1, wherein the electrical interface spacer lies in an electrical spacer plane and the biological interface spacer lies in a biological spacer plane, wherein the longitudinal axis of each needle electrode is normal to both of the electrical spacer plane and the biological spacer plane.

13. The array of claim 1, wherein each needle electrode of the plurality of needle electrodes is positioned less than 2 millimeters from another needle electrode.

14. A needle probe array comprising:
an interface board located at a proximal electrical interface region of the needle probe array, wherein the interface board defines a plurality of interface board openings within the interface board;
a biological interface spacer located at a distal biological interface region of the needle probe array, wherein the biological interface spacer defines a plurality of biological spacer openings extending within the biological interface spacer; and
a plurality of needle electrodes, wherein a first portion of each needle electrode of the plurality of needle electrodes is received in a corresponding opening of the plurality of interface board openings and a second portion of each needle electrode is received in a corresponding opening of the plurality of biological spacer openings, wherein each needle electrode of the plurality of needle electrodes extends along a longitudinal axis and comprises:
a head portion in the proximal electrical interface region and comprising at least three contact surfaces, wherein each of the at least three contact surfaces extends along a length parallel to the longitudinal axis, wherein the head portion further comprises non-contact surfaces between each of the at least three contact surfaces, wherein each of the at least three contact surfaces are configured to contact and electrically couple to a metalized surface of a corresponding interface board opening of the plurality of interface board openings and each of the non-contact surfaces are configured to lack contact with the metalized surface of the corresponding interface board opening when the head portion is received therein, and
a tip portion terminating the distal biological interface region and configured to penetrate into tissue.

15. The array of claim 14, further comprising an electrical interface spacer located at the proximal electrical interface region of the needle probe array, wherein the electrical interface spacer defines a plurality of electrical spacer openings extending within the electrical interface spacer, wherein the first portion of each needle electrode of the plurality of needle electrodes is received in a corresponding opening of the plurality of electrical spacer openings, wherein the plurality of electrical spacer openings are aligned with the plurality of interface board openings.

16. The array of claim 14, wherein each needle electrode of the plurality of needle electrodes defines a head portion end surface, wherein the head portion end surface is positioned a distance from an interface board first surface.

17. The array of claim 14, wherein the second portion of each needle electrode of the plurality of needle electrodes further comprises a biological spacer coupling portion configured to engage a surface defining the corresponding opening of the plurality of biological spacer openings in which it is received to maintain the needle electrode in a fixed position within the biological interface spacer.

18. The array of claim 17, wherein each needle electrode of the plurality of needle electrodes comprises an upper shaft between the head portion and the biological spacer coupling portion and a lower shaft between the biological spacer coupling portion and the tip portion, wherein the biological spacer coupling portion comprises a recessed region proximate the upper shaft and an expanded region between the recessed region and the lower shaft,
wherein a diameter of the recessed region is less than a diameter of the expanded region and a diameter of the upper shaft, wherein the diameter of the upper shaft is greater than the diameter of the expanded region, and
wherein each needle electrode of the plurality of needle electrodes extends through a corresponding biological spacer opening of the plurality of biological spacer openings such that the expanded region of the needle electrode contacts the surface defining the corresponding biological spacer opening to provide an interference fit between the needle electrode and the biological interface spacer.

19. A method of manufacturing a needle probe array comprising:
providing an electrical interface spacer defining a plurality of electrical spacer openings extending from an electrical spacer first surface to an electrical spacer second surface opposing the electrical spacer first surface;
positioning an interface board adjacent the electrical spacer first surface, wherein the interface board defines a plurality of interface board openings extending from an interface board first surface to an interface board second surface opposing the interface board first surface, wherein the interface board second surface is positioned facing the electrical spacer first surface, and further wherein the interface board is positioned such that the plurality of interface board openings align with the plurality of electrical spacer openings;
loading a plurality of needle electrodes into the plurality of interface board openings and then through the plurality of electrical spacer openings, wherein each needle electrode of the plurality of needle electrodes comprises:
a tip portion configured to penetrate into skin, wherein the tip portion passes through the plurality of interface board openings and the plurality of electrical spacer openings when loading the plurality of needle electrodes,
a head portion comprising at least three contact surfaces, wherein the head portion further comprises non-contact surfaces between each of the at least three contact surfaces, wherein the head portion of each needle electrode does not pass through the plurality of interface board openings when loading the plurality of needle electrodes, and
a biological spacer coupling portion located between the head portion and the tip portion;
applying force to the plurality of needle electrodes relative to the electrical interface spacer and interface board such that the head portions of the plurality of needle electrodes are moved within the plurality of interface board openings, wherein each of the at least three contact surfaces of the head portions of each of the plurality of needle electrodes contact and electrically couple to a metalized surface of a corresponding interface board opening of the plurality of interface board openings and the non-contact surfaces lack contact with the metalized surface of the interface board opening;
providing a biological interface spacer defining a plurality of biological spacer openings extending from a biological spacer first surface to a biological spacer second surface opposing the biological spacer first surface;
inserting the tip portions of the plurality of needle electrodes through the plurality of biological spacer openings; and
applying force to the plurality of needle electrodes relative to the biological interface spacer such that the biological spacer coupling portion of each needle electrode is fixed at a location within the biological interface spacer, wherein the biological spacer second surface is positioned a distance from the tip portions of the plurality of needle electrodes.

20. The method of claim 19, wherein applying force to the plurality of needle electrodes relative to the electrical interface spacer and interface board comprises applying force at the head portion of each needle electrode of the plurality of needle electrodes, wherein the head portion comprises a taper region to facilitate movement of the head portion into the plurality of interface board openings when the force is applied.
